(19) United States
(12) Patent Application Publication
(10) Pub. No.: US 2012/0141601 A1
(43) Pub. Date: Jun. 7, 2012

(54) DIETARY SUPPLEMENT, FEED AND PHARMACEUTICAL COMPOSITION CONTAINING CHIA SEED AND MACA, AND PREPARATION METHOD THEREOF

(75) Inventors: Younjin Kim, Yongsan-gu (KR); Hojun Kim, Yongsan-gu (KR)

(73) Assignee: Hanyang Patent Firm, Seoul (KR)

(21) Appl. No.: 13/390,064

(22) PCT Filed: Aug. 11, 2010
(86) PCT No.: PCT/KR10/05278

§ 371 Date: Feb. 10, 2012

(30) Foreign Application Priority Data
Aug. 11, 2009 (KR) 10-2009-0073702
Aug. 11, 2010 (KR) 10-2010-0077099

(51) Int. Cl. A61K 36/537; A61P 1/00; A61P 1/16; A61P 15/00

(52) U.S. Cl. 424/643; 424/746; 424/650; 424/702

(57) ABSTRACT

Disclosed are a composition comprising chia seeds and maca, a dietary supplement or food additive, a feed and a pharmaceutical composition, all being based on the composition, and a method for preparing the dietary supplement, feed or pharmaceutical composition. The dietary supplement or food additive, the feed or feed additive, and the pharmaceutical composition exhibit can bring about excellent improvement in weight loss, sexual, gastrointestinal or hepatic function, male sex hormone release and endurance. In addition to a combination of chia seeds and maca, the composition, the food and the like may comprise optionally germanium, selenium or dietary zinc, and they are useful for the potentiation of sexual, gastrointestinal or hepatic functions, which leads to an improvement in weight loss, fatigue resistance, endurance, blood circulation, blood cholesterol level, anti-inflammation, immunity, sex hormone release, skin care, hangover cure, nicotine removal, breath care, body smell, invigoration and sterility.

### BACKGROUND OF THE INVENTION

1. Technical Field

The present invention relates to a composition comprising chia seeds and maca. Also, the present invention relates to a dietary supplement, a feed and a pharmaceutical composition, all being based on the composition, and a method for preparing the dietary supplement, feed or pharmaceutical composition. The dietary supplement, food additive, feed or feed additive, and pharmaceutical composition exhibit can bring about excellent improvement in weight loss, sexual, gastrointestinal or hepatic function, increasing male sex hormone and endurance.

2. Description of the Related Art

Today, people take a high interest in health foods, with the trend in food taste being towards sophistication, diversification and westernization. However, a westernized dietary life and stress from excessive work loads have necessarily exposed people to various adult diseases including liver diseases, immune diseases, motor disturbance, and renal dysfunction. Further, men under excessive stress are likely to suffer from a lack of virility and sexual dysfunction as well as neural, endocrinal, and cardiovascular disorders.

On the whole, muscular endurance is the ability to remain active for a long period of time and is a state in which the blood hemoglobin concentration, the metabolic activity of mitocondria in muscle cells, vasodilation, and the activation of cardiaomuscular metabolism are involved. It is known that muscular endurance can be enhanced by recovering muscle contractility by preventing the accumulation of fatigue substances such as inorganic phosphate, lactate, etc., from intramuscular accumulation and the rapid regeneration of products of energy metabolism. Sexual function in males is a wide concept that encompasses sexual desire, erectility, ejection and orgasm. Abnormality in the neural, endocrinal or cardiovascular system may cause impotence, sexual desire disorder, erectile dysfunction, ejaculatory disturbance, and/or orgasmic disturbance, resulting in male libido dysfunction. Particularly, a high-fat diet, often eaten by people these days, increases serum cholesterol levels, causing dysfunctions in the cardiovascular system and the liver, and thus has been pointed to, together with stress, as one of the main causes of sexual dysfunction. What is more serious, the uptake of a high-fat diet may result in decreased endurance in daily life.

Statistics showed that 20˜60% of adult men suffer from sexual dysfunction, with the attack rate increasing with age. Only ten years ago, sexual dysfunction was regarded as a disease of adult people, but medical advances have revealed that sexual dysfunction is attributed to vascular, neural or endocrinal diseases, diabetes, hypertension, drug intake, etc. in about 50% of the patients.

With regard to the physiology of penile erection, active research has been performed into substances that have an influence on the relaxation of the corpus cavenosum. For example, intracorporal injection therapy with papaverine, phentolamine, and prostaglandin E1 is used clinically. Papaverine is an opium alkaloid that causes direct smooth muscle relaxation when injected in penile tissues while phentolamine is an adrenergic antagonist. These substances are, however, restricted from being used because of such side effects as pain, priapism, cavernous fibrosis, etc. Trazodone and yohimbine have also been developed as oral drugs. However, their pharmaceutical mechanisms remain still unclear. Sildenafil, a phosphodiesterase type 5 (PDE5) inhibitor, was developed, but suffers from the disadvantage of temporary performance, being expensive, and causing headaches, hypertension and myocardial infarction. It is therefore necessary to develop an effective therapeutic agent for sexual dysfunction that potentiates the erectile function of the body itself and is safe. Recently, extensive attention has been paid to the development of agents useful for the treatment of sexual dysfunction that can increase the production of NO (nitric oxide) and cGMP in the smooth muscle of the corpus cavenosum to cause the penile tissue to undergo strong relaxation.

Many invigorants based on the prescriptions of herb medicines have been introduced for the therapy of sexual dysfunction. However, medical research into the pharmaceutical mechanisms of the invigorants has not actually been done. Although many single ingredients have been isolated from herbs and formulated, little is known about their pharmaceutical effects on sexual function or endurance. The natural materials known to date to enhance endurance include wheat germ, rice bran, and sugar cane. A report showed that octacosanol, present in a trace amount in the peels of apple and grape, is involved in the physiological functions including the enhancement of endurance, the regulation of serum cholesterol levels, the activation of cardiopulmonary function and metabolism, and the regulation of the storage of glycogen in the muscle and liver. Citrulline is known as an ergogenic nitric oxide stimulator that stimulates muscle blood flow. Ginseng, especially red ginseng, is found to scavenge hydroxy radicals and have potent antioxidative activity, which is used to enhance sexual function and erectility. These materials have been actively studied to develop agents useful in the treatment of sexual dysfunction. In spite of extensive research, effects found in such natural materials have fallen short of Viagra from the point of view of sexual function. Moreover, the patients bear the burden of increasing medical fees as well as suffering from the disease. There is therefore a pressing need for an agent that is effective for the therapy of sexual dysfunction and for the enhancement of endurance and that can be useful in a wide spectrum of clinical fields.

Further, these efficacies are necessary for animals such as livestock as well as humans Thus, feed or feed additives showing the same effects are also in demand.

There are patent publications that address the enhancement of sexual function and endurance induced by, for example, the functional eggs from chickens fed with a culture of wild-ginseng (Korean Pat. Appl'n No. 2005-0115891, the Korea Food Research Institute), red ginseng and Acanthopanax senticosus micrococcin (Korean Pat. Appl'n 1997-0024211), glutamine peptide and chlorella growth factor (Korean Pat. Appl'n 1998-0040319), propolis extract powder (Korean Pat. No. 10-0329010), and octacosanol (Korean Pat. 10-0329011).

### SUMMARY OF THE INVENTION

### Technical Problem

Leading to the present invention, intensive and thorough research into materials that can bring about an improvement in sexual dysfunction and endurance conducted by the present inventors aiming to overcome the problems encountered in the prior art, resulted in the finding that a combination of chia seed and maca is very effective for enhancing the health of both humans and livestock, and it is therefore an object of the present invention to provide a dietary supplement and food additive comprising a combination of chia seed and maca as active ingredients.

It is another object of the present to provide a feed or feed additive comprising a combination of chia seed and maca as active ingredients.

It is a further object of the present invention to provide a pharmaceutical composition comprising a combination of chia seed and maca as active ingredients.

It is still a further object of the present invention to provide a method for preparing the dietary supplement or food additive, the feed, or the pharmaceutical composition.

### Technical Solution

In order to accomplish the objects of the present invention, there is provided a dietary supplement, food additive, feed or feed additive, comprising chia seeds and maca at a weight part ratio of 100:5˜250.

Chia seeds and maca may be used as they are or after being processed into powders or extracts. For example, extracts from chia seeds and maca may be prepared in a solvent selected from the group consisting of water, ethanol and a mixture thereof.

Also, the dietary supplement, food additive, feed or feed additive may further comprise a mineral selected from the group consisting of germanium, selenium, dietary zinc, and a mixture thereof.

The content of the mineral in the composition is on the order of 0.1 to 10 parts by weight based on 100 parts by weight of chia seed.

In addition, the mineral is preferably an extract of the solvent selected from the group consisting of, but not limited to, water, a lower alcohol, ethyl acetate, aromatic hydrocarbons, chlorohydrocarbons, and a mixture thereof.

As for the food additive or feed additive, it may be used in an amount of from 0.001 to 5% by weight and preferably in an amount of from 0.5 to 2.5% by weight based on the total weight of the food or feed to which it will be added.

In an embodiment, the pharmaceutical composition of the present invention may be a composition for the enhancement of sexual, gastrointestinal or hepatic function, comprising as an active ingredient an extract from a combination of a weight ratio of 100:5˜250 chia seed:maca in a solvent selected from the group consisting of water, ethanol and a mixture thereof.

In another embodiment, the method for preparing the dietary supplement, the food additive, the feed, the feed additive, or the pharmaceutical composition in accordance with the present invention may comprise, but is not limited to, the following steps:

(A) mixing 100 parts by weight of chia seeds with 5˜250 parts by weight of maca in 500˜7000 parts by weight of a solvent selected from the group consisting of water, ethanol and a mixture thereof, based on 100 parts by weight of chia seeds;

(B) heating the mixture of the chia seed, the maca in the solvent at a temperature of 30˜60° C. for 2˜6 hours to afford an extract; and

(C) subjecting the extract to filtration and concentration to dryness.

The method may further comprise the step (D) of pulverizing the chia seeds and the maca into a powder with a mean particle size of 0.3˜2 μm prior to the step (A).

Also, the pharmaceutical composition may further comprise a pharmaceutically acceptable additive selected from the group consisting of an excipient, a supplement, a diluent, an isotonic agent, a preserver, a lubricant, a dilution aid, and a mixture thereof.

Also, the pharmaceutical composition may further comprise an ingredient selected from the group consisting of Viagra, Levitra, Cialis, a heavy drug, Zydena, green tea, a nucleic acid complex, an alga-derived physiologically active substance, a squeeze of oyster mushroom, a zhibeisan extract, micrococin and a mixture thereof.

In another embodiment, a function of the pharmaceutical composition may be activating dermal cells in addition to enhancing a sexual, gastrointestinal or hepatic function.

In another embodiment, the pharmaceutical composition may be for curing hangover.

In another embodiment, the pharmaceutical composition may be for removing nicotine.

In another embodiment, the pharmaceutical composition may be for breath care.

In another embodiment, the pharmaceutical composition may be for treating erectile dysfunction.

In another embodiment, the pharmaceutical composition may be for treating sterility.

In another embodiment, the pharmaceutical composition may be for reducing body weight.

In another embodiment, the pharmaceutical composition may be for providing fatigue resistance.

In another embodiment, the pharmaceutical composition may be for enhancing endurance.

In another embodiment, the pharmaceutical composition may be for enhancing blood circulation.

In another embodiment, the pharmaceutical composition may be for reducing blood cholesterol levels.

In another embodiment, the pharmaceutical composition may be for inhibiting inflammation.

In another embodiment, the pharmaceutical composition may be for potentiating immunity.

In another embodiment, the pharmaceutical composition may be for stimulating the release of sex hormone.

In another embodiment, the dietary supplement or the food additive may be used to enhance a gastrointestinal, sexual or hepatic function.

In another embodiment, the dietary supplement or the food additive may be used for weight loss, anti-fatigue, endurance enhancement, blood circulation enhancement, the reduction of blood cholesterol levels, anti-inflammation, immunity potentiation, sex hormone secretion, skin car, hangover cure, nicotine removal, breath care, invigoration or the treatment of sterility.

In another embodiment, the feed or the feed additive may be used to enhance a gastrointestinal, sexual or hepatic function.

In another embodiment, the feed or the feed additive may be used for weight loss, anti-fatigue, endurance enhancement, blood circulation enhancement, the reduction of blood cholesterol levels, anti-inflammation, immunity potentiation, sex hormone increase, skin care, hangover cure, nicotine removal, breath care, invigoration or the treatment of sterility.

### Advantageous Effects

Chia seeds, used as an ingredient of the dietary supplement, etc. in accordance with the present invention, is rich in dietary fiber, vegetable fat including omega-3 fatty acid, amino acids and minerals, but free of cholesterol, so that they are used as a stamina food for enhancing endurance, a food for health and skin care, and even a diet food. However, the long-term intake of chia seeds causes enervation and fatigue due to weight loss, and may even be accompanied by adverse effects such as diarrhea.

In this invention, the present inventors found that when used in combination with maca, chia seeds exert their useful functions, along with the additional efficacy attributable to maca, while avoiding the problems.

The dietary supplement, the food additive, the feed, the feed additive or the pharmaceutical composition according to the present invention comprises a combination of chia seeds and maca, particularly in the form of a powder or an extract, as an active ingredient, and optionally germanium, selenium or dietary zinc can be added, and they are useful for the potentiation of sexual, gastrointestinal or hepatic functions, which leads to an improvement in weight loss, fatigue resistance, endurance, blood circulation, blood cholesterol level, anti-inflammation, immunity, sex hormone release, skin care, hangover cure, nicotine removal, breath care, body smell, invigoration and sterility.

### BRIEF DESCRIPTION OF THE DRAWINGS

FIG. 1 is a graph showing changes in body weight with the administration of chia seeds and maca.

FIG. 2 is a graph showing the effect of chia seeds and maca on blood pressure.

FIG. 3 is a graph showing the effect of chia seeds and maca on endurance as analyzed in a swimming experiment.

FIG. 4 is a graph showing the effect of chia seeds and maca on locomotor activity as analyzed by an open field test.

FIG. 5 is of photographs showing the effect of chia seeds and maca on the trajectory of rats in an open field test.

FIG. 6 is a graph showing the effect of chia seeds and maca on space perception as analyzed by a rotarod performance test.

FIG. 7 is a graph showing changes in blood alcohol concentration with the administration of chia seeds and maca.

FIG. 8 is a graph showing the effect of chia seeds and maca on alcohol dehydrogenase activity.

FIG. 9 is a graph showing the effect of chia seeds and maca on aldehyde dehydrogenase activity.

FIG. 10 is a graph showing the effect of chia seeds and maca on the level of testosterone in whole blood.

FIG. 11 is a graph showing the effect of chia seeds and maca in the level of testosterone in peripheral blood taken from the tail vein.

FIG. 12 is a graph showing the effect of chia seeds and maca on the level of high-density lipoproteins in whole blood

### DESCRIPTION OF THE PREFERRED EMBODIMENTS

Reference now should be made to the drawings, throughout which the same reference numerals are used to designate the same or similar components. Below, a description will be given of preferred embodiments of the present invention in conjunction with the accompanying drawings. Throughout the accompanying drawings, the same reference numerals are used to designate the same or similar components. It should be apparent to those skilled in the art that although many specified elements such as concrete components are elucidated in the following description, they are intended to aid the general understanding of the invention and the present invention can be implemented without the specified elements. Further, in the description of the present invention, when it is determined that the detailed description of the related art would obscure the gist of the present invention, the description thereof will be omitted.

An extensive study, conducted by the present inventors, on natural substances that are therapeutically effective at treating various adult diseases including liver diseases, motor dysfunction, renal dysfunction, and particularly with respect to degenerative diseases of the nervous system, the endocrinal system and the cardiovascular system, culminated in the surprising finding that chia seeds and maca, in the form of either a powder or an extract, have the activity of inducing weight loss, enhancing sexual function, stimulating the release of male sex hormones, increasing endurance and avoiding renal dysfunction, which culminated in the present invention.

Chia seeds, useful as a main ingredient in the composition of the present invention, are characterized by the following traits and pharmaceutical efficacies.

Chia seeds are seeds of a species of flowering plant in the mint family, Lamiaceae (Salvia hispanica or Salvia columbariae), native to Mexico and South America. Ten hundred years ago, people in these regions have eaten chia seeds as a food for enhancing endurance, stamina, health and skin care. Chia seeds are consumed in Mexico, the south-western region of U.S., and South America, but have not yet been widely known to Asia or Europe.

When soaked in water, chia seeds become gelatinous in texture so that their volume increases by ten fold. They also increase in volume even in the gastric fluid, giving the sensation of being full, and thus are used as a diet food. In addition, chia seeds inhibit the uptake of lipids in vivo, contributing to weight loss.

Chia seeds are 28% dietary fiber, which is more than its content in lettuce, spinach, and agar. Dietary fibers, although digestible in the human body, affect the digestive intake of other nutrients with the passage thereof from the stomach to the small and the large intestines due to their physicochemical properties such as water retention, cation exchange, gelling, and absorption. A diet containing an excessive content of dietary fibers is likely to pass through the digestive organ so rapidly that its digestion and absorption are insufficient, which may result in weight loss. Further, an excess of dietary fiber may cause diarrhea or dehydration because they are excreted in a hydrated form.

Of chia seeds, about 32% is vegetable fat, over 60% of which is accounted for by omega-3 fatty acids including linoleic acid. The content of omega-3 fatty acids in chia seeds is higher than that in other omega-3 fatty acid sources such as fish and flax.

Also, chia seeds are a natural health food abundant in 18 amino acids including glutamine, arginine, asparagine, leucine, valine and alanine, and absolutely free of cholesterols. Further, they have high contents of contains minerals such as calcium, phosphor, magnesium, potassium, iron, zinc and copper, and contain natural antioxidants.

In addition, the dietary fibers of chia seeds correspond to a mixture of polysaccharides with respective molecular weights of 74674, 21722, 8066, 2038, and 1050. These low- and average molecular weight polysaccharides in mixture account for the porous skin of chia seeds, and retain 5- to 40 times their volume of water therein. When soaked in water for 20˜30 min, chia seeds become gelatinous with many kinds of polysaccharides existing therein.

Although chia seeds are advantageous as mentioned above, they may cause weight loss-induced side effects such as enervation and fatigue when ingested for a long period of time such as several weeks. Further, around 10% of the people who take chia seeds suffer from diarrhea as mentioned above.

Out of the experiments in which the chia seeds are combined with various ingredients, a combination of chia seeds and maca was found to exhibit the beneficial efficacies of chia seeds without the problems in addition to exerting additional efficacies.

Maca, acting as an essential element in the present invention, is also taxonomically classified as Lepidium meyenii Walp, and is used as a medicinal herb for invigoration and fatigue resistance. The form of the flower of wild maca is similar to that of roses and its dried roots are edible. The plant, native to the high Andes, is found at higher than 4000 m above sea level, and has grown in the severe weather before the Inca Age. Maca, which naturally grows under such hard environmental conditions as strong daylight, overnight low temperatures, low atmosphere, a high wind, etc., is used as a food for human and livestock due to its rich nutrients. Its roots and tubers look like those of potatoes. One thin stem from the tuber extends upward to the extent of about 5 cm high from the ground. Thus, wild maca is usually found for its leaves which have grown from the stem on the ground. Maca leaves look somewhat shriveled. As many as 11 kinds of maca are known thus far in Peru. They have the various colors of bright yellow, deep purple, turbid pink, pastel pink, etc., but are not brilliant so that they look like they are withering.

Maca, also called the ginseng of Andes, is known to potentiate sexual functions such as virility. Reports show that the amino acids lysine and arginine, found in maca, activate not only the genital organs to prevent sterility attributed to, for example, ovarian dysfunction, but also normalize the release of female sex hormones to mitigate menstrual pain or menstrual irregularity. Maca is also known to improve the menopausal symptoms attributed to rapid hormonal decrease.

In addition, maca is abundant in ‘alkaloid,’ which is effective for the treatment of stress-induced impotence. It also contains a steroid associated with male hormones which stimulate the production of spermatozoa and ova, and dextrin which makes smooth blood flow through the penile artery, and other physiologically active substances, such as anthocyanin, saponin, terpenoid, etc. When these ingredients act in vivo over a long period of time, penile dysfunction may be spontaneously cured. In contrast to synthetic cures for penile dysfunction, such as Viagra, the natural herb maca causes no side effects.

In brief, maca has the physiological activity of increasing sperm count and motility for men with penile dysfunction or weak virility and of stimulating the release of estrogen for women to sterility and menopausal symptoms. Further, the publication issued by the KFDA describes that maca is effective for the control of female sex hormonal release, the improvement of fertility, invigoration, immunopotentiation, and the improvement of anemia, chronic fatigue syndrome, enervation, climacterium, and menstrual pain.

When a combination of maca and chia seeds is ingested, the side effects of chia seed alone, such as enervation and fatigue attributed to weight loss, and diarrhea, are remarkably diminished. In detail, the combination of chia seed and maca increases endurance, improves basal metabolic rates, and stimulates the synthesis of male hormones. Further, it is advantageous for liver function. Therefore, the composition of the present invention may be used as a cure for penile dysfunction or an invigorant without such side effects as enervation, fatigue, and body temperature decrease. As a result, a dietary supplement or pharmaceutical composition which can improve sexual functions, endurance and liver functions in people or livestock can be provided.

Maca and chia seeds may be ingested as they are, or in the form of powders. Alternatively, they are used in the form of extracts. In this regard, they may be extracted individually or together. In the former case, the individual extracts may be combined before ingestion. In another embodiment, the extraction may be done together with other necessary herbs.

### EXAMPLES

### Example 1

A mixture of 10 g of chia seeds and 5 g of maca was pulverized into particles with a size of 0.5 μm, and distilled water was added to the mixture to form a final volume of 1000 mL, followed by extraction in a bath at 45° C. for 3 hours. The solution was passed through gauze and the filtrate was lyophilized to afford 2 g of powder.

### Example 2

The same procedure as in Example 1 was repeated, with the exception that a mixture of 5 g of chia seed and 10 g of maca was used and the lyophilization was not conducted, to give a liquid filtrate.

### Example 3

The same procedure as in Example 2 was repeated, with the exception that a mixture of 10 g of chia seeds and 5 g of maca was used

### Example 4

The same procedure as in Example 2 was repeated, with the exception that a mixture of 100 g of chia seeds and 50 g of maca was used

### Example 5

The same procedure as in Example 1 was repeated, with the exception that a mixture of 25 g of chia seeds and 50 g of maca was used

### Example 6

The same procedure as in Example 1 was repeated, with the exception that a mixture of 75 g of chia seeds and 50 g of maca was used

### Example 7

The same procedure as in Example 1 was repeated, with the exception that a mixture of 100 g of chia seeds and 50 g of maca was used

### Example 8

3 g of maca was pulverized into particles with a size of 1 μm and mixed with 10 g of chia seeds. In this regard, the chia seeds were used as they were.

### Example 9

50 g of maca was pulverized into particles with a size of 0.3 μm and mixed with 50 g of chia seeds. In this regard, the chia seeds were used as they were.

### Example 10

10 g of chia seeds and 5 g of maca were separately pulverized into particles with a size of 0.5 μm, and distilled water was added to each herb powder to form a final volume of 1000 mL, followed by extraction in a bath at 45° C. for 3 hours to give extracts. The extracts were mixed together and passed through gauze and the filtrate was lyophilized to afford 2 g of powder.

### TEST EXAMPLES

### Test Example 1

### Breeding and Change in Weight of Rats

For use in examining the ability of the composition of the present invention to enhance liver function and endurance, Sprague-Dawley male rats, each weighing about 200 g, were purchased from Orientbio. During experiments, they were maintained at a temperature of 22±2° C. and a relative humidity of 50±10%, with an automated light-dark cycle of 12:12 hours. The rats were randomly assigned to groups of six. The control group and test groups, but not the normal group, were orally administered with 25% alcohol every day for 5 weeks. The body weight of all of the rats was measured every three days to monitor weight changes per week. In this course, the effects of chia seeds and maca (extracts), used as the active ingredient in the composition of the present invention, were examined. When the extract powder prepared in Example 1 was used, its content in the feedstuff for the rats was maintained at 0.5 to 2.5% by weight.

After 5 weeks, the body weights were measured to be 380.5±13.3 g in the normal group administered with neither alcohol nor the extract, 350.2±11.2 g in the control group, administered with alcohol, but not with the extract, 340.2±5.2 g in the treatment group administered with alcohol and a chia seed extract, 317.1±3.1 g in the treatment group administered with alcohol and a maca extract, and 315.1±7.2 g in the group administered with alcohol and a mixture of chia seed and maca extracts. Therefore, the body weight of the groups administered with an extract from chia seeds and/or maca decreased by 11 to 18% (FIG. 1).

### Test Example 2

### Measurement of Blood Pressure

Excessive stress or alcohol intake increases both the blood circulation rate and the heart rate, thus raising blood pressure. The rats in the control group and test groups, but not in the normal group, were administered with 25% alcohol. The blood pressures of all of the rats were measured. Mean values were recorded on the Blood Pressure Monitoring System (BP-2000 Series II, USA). For the measurement of tail-cuff blood pressure, the Sprague-Dawley male rats (Orientbio) in the normal, the control group and the test groups were placed for 20 min in restrainers in a preheating chamber and their body temperature was maintained at 34° C. by the preheating chamber for 10 min The tail-cuff sensor was placed on the tail and attached to the restrainer. After acclimation for 5 min, blood pressures were measured. Blood pressures were determined as mean values of four measurements while the normal mean blood pressure was fixed at 33.

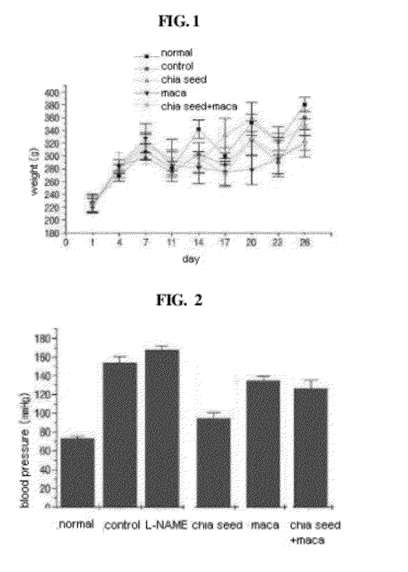

To investigate the anti-hypertensive effects of chia seeds and maca (extract), used as active ingredients in the composition of the present invention, blood pressure measurements were analyzed. The blood pressure was recorded at 73.5±2.0 mmHg for the normal group administered with neither the alcohol nor the extract, at 153.6±6.8 mmHg for the control group administered with alcohol, but not with the extract, at 167.8±3.9 mmHg for the treatment group administered with alcohol and L-NAME (nitro-L-arginine methyl ester. Conc. 1 mg/ml. 1 ml/rat), at 95.5±5.9 mmHg for the treatment group administered with alcohol and chia seeds (chia seeds 10 g/feedstuff 1 kg/1 day/1 kg), at 134.8±4.6 mmHg for the treatment group administered with alcohol and maca (maca 5 g/feedstuff 1 kg/1 day/1 kg), and at 126.6±8.9 mmHg for the group administered with alcohol and a mixture of chia seeds and maca (chia seeds 10 g+maca 5 g/feedstuff 1 kg/1 day/1 kg) (FIG. 2). As can be seen, the blood pressure decreased in the groups co-administered with chia seeds and maca, compared to the control group and the L-NAME-administered group.

### Test Example 3

### Measurement of Endurance

Sprague-Dawley male rats (Orientbio) were divided into groups of six which were then administered for 26 days with neither alcohol nor the extracts for the normal group, with alcohol but not with extracts for the control group, with alcohol and 1% by weight octacosanol, with alcohol and 1% by weight chia seeds, with alcohol and 0.5% by weight maca, and with alcohol and a mixture of 1% by weight chia seeds and 0.5% by weight maca, respectively. Typical feedstuff was fed, together with water, to the normal group and, together with 25% alcohol, to the control group while the feedstuff for the other groups contained the test substances as described above.

A swimming experiment was conducted in a transparent acryl bath (with a dimension of length 80 cm×width 50 cm×depth 90 cm) ⅔ filled with water maintained at 24° C. The Sprague-Dawley male rats (Orientbio) were allowed to freely swim with a weight equivalent to 8˜10% of their body weight suspended from the abdomen thereof. Measurements were made of the time it took for the rat to swim to a point at which the head was submerged for 10 sec or longer below the water surface (Hong, S. G., J. Kor. Food Sci. Nutr. 32(7), 1076 (2003)).

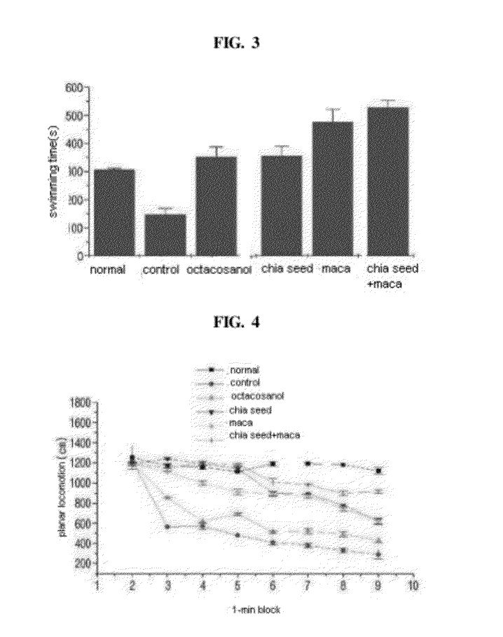

To investigate the effects of chia seeds and maca (extract), used as active ingredients in the composition of the present invention, on endurance, measurements of the swing time were analyzed. The swimming time was measured to be 306.33±6.21 sec for the normal group, but 147.16±21.71 sec for the control group, which was equivalent to about 46% of that of the normal group. In contrast, 352.66±34.69 sec was measured for the octacosanol-administered group, 355.66±34.62 sec for the chia seed-administered group, 476.00±45.56 sec for the maca-administered group, and 527.83±25.57 sec for the group co-administered with chia seeds and mace, which were increased by 2.4, 2.4, 3.2 and 3.5 times, compared to the control group. As apparent from the data of the swimming experiment, endurance was enhanced by octacosanol, chia seeds, maca, and a combination of chia seeds and maca, and was increased by the most by, inter alia, the mixture of chia seeds and maca (FIG. 3, Table 1).

TABLE 1
Endurance Test with Swimming in Bath

| Group | Swimming time (sec) |
|---|---|
| Normal | 306.33 6.21 |
| Control | 147.16 ± 21.71 |
| Octacosanol administered | 352.66 ± 34.69 |
| Chia seed administered | 355.66 ± 34.62 |
| Maca administered | 476.00 ± 45.56 |
| Chia seed and maca | 527.83 ± 25.57 |
| co-administered | |

### Test Example 4

### Locomotor Measurement

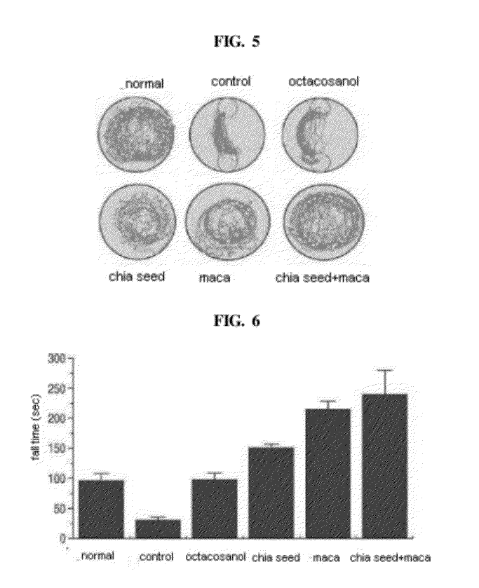

The locomotor activities of the groups set in Test Example 3 were measured in an open field test chamber (38.0 cm×26.0 cm×42.0 cm; LETICA, SPAIN). Horizontal activity was recorded by an IR detector for 10 min To investigate the effects that chia seeds and maca (extract), used as active ingredients in the composition of the present invention, had on locomotor activity, measurements of the horizontal activity were analyzed. The horizontal activity was 1179.2±21.2 cm for the normal group, but 30±21.2 cm for the control group, which was decreased by about 75% compared to the normal. The normal group was observed to actively locomote across the plane whereas the rats of the control group mostly aggregated at the central region. In contrast, the horizontal activity was recorded to be 240.2±12.3 cm for octacosanol-administered group, 510.1±21.3 cm for the chia seed-administered group 718±11.3 cm for the maca-administered group, and 891±17 cm for the group co-administered with chia seeds and maca, which were increased by 8, 17, 24, and 30 times, compared to the control group. As apparent from the data of the open field test, locomotor activity was enhanced by octacosanol, chia seeds, maca, and a combination of chia seeds and maca, and most increased, inter alia, by the mixture of chia seeds and maca (FIGS. 4 and 5).

### Test Example 5

### Space Perception Test

The groups set in Test Example 3 were measured for space perception activity by the rotarod performance test which was conducted for 5 min for each rat with the cylinder (380×260×420 mm) of rotarod LE8300 (LETICA, SPAIN) rotating at a speed of 4 to 40 rpm. Cases in which the rats fell on the ground within 20 sec after starting were excluded from the measurement.

To investigate the effect of chia seeds and maca (extract), used as active ingredients in the composition of the present invention, on space perception and endurance, measurements of the rotarod performance test were analyzed. Measurements were made of the time that the rats stayed on the rotating cylinder and were found to be 96.83±11.42 sec for the normal group, but 31.16±4.53 sec for the control group, which was decreased by about 32%, compared to the normal group. In contrast, the time was 98.16±11.35 sec for octacosanol-administered group, 151.50±5.08 sec for the chia seed-administered group, 215.33±13.14 sec for the maca-administered group and 240.66±39.30 sec for the group co-administered with chia seeds and maca, which were increased by 3, 5, 7, and 8 times, respectively, compared to the control group. As apparent from the data of the rotarod performance test, space perception and endurance were enhanced by octacosanol, chia seeds, maca, and a combination of chia seeds and maca, and most increased, inter alia, by the mixture of chia seeds and maca (FIG. 6, Table 2).

TABLE 2
Rotarod Performance Test for Space
Perception and Endurance

| Group | Swimming time (sec) |
|---|---|
| Normal | 96.83 ± 11.42 |
| Control | 31.16 ± 4.53 |
| Octacosanol administered | 98.16 ± 11.35 |
| Chia seed administered | 151.50 ± 5.08 |
| Maca administered | 215.33 ± 13.14 |
| Chia seed and maca | 240.66 ± 39.30 |
| co-administered | |

### Test Example 6

### Measurement of Blood Alcohol Concentration

Blood alcohol concentrations were measured after the rats were administered 25% alcohol every day for 30 days. In this regard, blood samples were taken from the tail vein of the rats of the test groups set in Test Example 3 at 6 P.M. every five days, and blood alcohol concentrations were determined using an alcohol tester (Asia Commerce Co. Ltd., Model No. LION SD-400).

To investigate the effect of chia seeds and maca (extract), used as active ingredients in the composition of the present invention, on the degradation of blood alcohol, measurements of the blood alcohol concentration were analyzed. The blood alcohol concentration was 0% for the normal group and 0.06% for the control group. In contrast, it was 0.034% for octacosanol-administered group, 0.024% for the chia seed-administered group, 0.019% for the maca-administered group and 0.016% for the group co-administered with chia seeds and maca, which were decreased by 55%, 60%, 69%, and 74%, respectively, compared to the control group. As is apparent from the data of the blood alcohol test, it was concluded that the ingredients of the present invention activate the alcohol degradation function of the liver (FIG. 7).

### Test Example 7

### Effect on Alcoholysis Enzyme Activity

In the body, ethanol is oxidized to acetaldehyde, which is toxic to the body, by alcohol dehydrogenase and then, further converted to acetate, non-toxic, by aldehyde dehydrogenase.

In order to investigate the effect of the composition of the present invention on alcoholysis enzymes, the rats of the test groups set in Test Example 3 were anesthetized with chloroform and subjected to ventrotomy to excise the liver. The liver tissue was homogenized in 10 volumes of 100 mM sodium phosphate buffer, pH 7.4, using a homogenizer and then disrupted with an ultrasonicator. Centrifugation at 700 G for 10 mM removed nuclei and cell debris. After centrifugation at 9,000 G for 20 min, the supernatant was measured for hepatic alcohol dehydrogenase and aldehyde dehydrogenase activity. The protein concentration of the supernatant was determined using a BCA kit and used to normalize the amount of liver tissue between test groups. The activity of alcohol dehydrogenase was determined by absorbance at 340 nm after 10 μl of the liver homogenate was added to 50 mM sodium phosphate buffer (pH 7.4) containing 1 mM NAD+, 5 μM rotenone (mitochondrial NADPH oxidase inhibitor), and 5 mM ethanol and incubated for 5 mM As for aldehyde dehydrogenase, its activity was also determined by absorbance at 340 nm 10 μl of the liver homogenate which has been incubated for 5 mM in 50 mM sodium phosphate buffer (pH 7.4) containing 1 mM NAD+, 5 μM rotenone, 0.1 mM pyrazole (ALD inhibitor), 5 mM propanal, and 2 mM 2-mercaptoethanol.

To investigate the effect of chia seeds and maca (extract), used as active ingredients in the composition of the present invention, on the degradation of blood alcohol, measurements of the alcohol dehydrogenase activity were analyzed. Alcohol dehydrogenase activity was found to be 13.8±1.4 mmole/min/mg protein for the normal group, 4.7±0.7 mmole/min/mg protein for the control group, 8.9±0.8 mmole/min/mg protein for the octacosanol-administered group, 16±1.2 mmole/min/mg protein for the chia seed-administered group, 14.5±1.0 mmole/min/mg protein for the maca-administered group, and 19±1.2 mmole/min/mg protein for the group co-administered with chia seeds and maca. As shown, the co-administration of chia seeds and maca induced the highest activity of alcohol dehydrogenase. It is thought that the increase in alcohol dehydrogenase activity, compared to the control group, is because of the fact that chia seeds and maca enhance hepatic function.

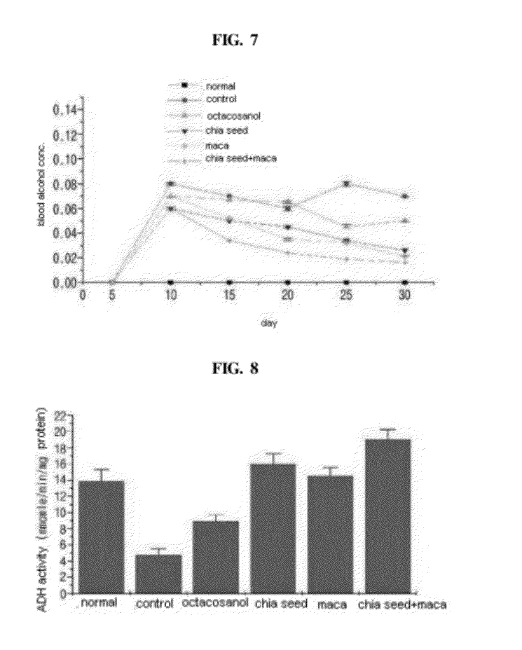

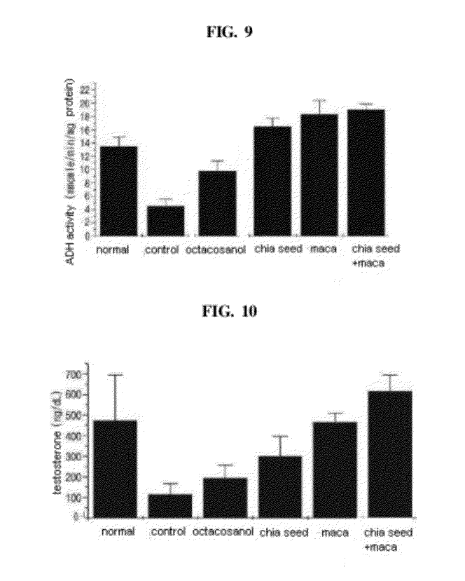

In addition, aldehyde dehydrogenase activity was measured at 13.5±1.3 mmole/min/mg protein for the normal group, 4.5±1.0 mmole/min/mg protein for the control group, 9.8±1.4 mmole/min/mg protein for the octacosanol-administered group, 16.5±1.2 mmole/min/mg protein for the chia seed-administered group, 18.3±2.0 mmole/min/mg protein for the maca-administered group, 19±0.8 mmole/min/mg protein for the group co-administered with chia seeds and maca. Likewise, the aldehyde dehydrogenase activity was most greatly increased in the group co-administered with chia seeds and maca. This data, therefore, allows the conclusion to be drawn that chia seeds and maca have potent hangover remedy activity (FIGS. 8 and 9).

### Test Example 8

### Measurement of Male Sex Hormone and High-Density Lipoprotein Levels

After the rats of the test groups set in Test Example 3 were administered with 25% alcohol every day for 25 days, blood samples were taken from the tail vein and measured for testosterone level using enzyme immunoassay (Roche, USA). The levels of testosterone and high-density lipoproteins in the whole blood taken from the heart were measured in Eone Reference Laboratory.

To investigate the effect of chia seeds and maca (extract), used as active ingredients in the composition of the present invention, on testosterone activity, the levels of testosterone in whole blood were analyzed. It was recorded at 473.0±223.0 ng/dL for the normal group, at 115.1±54.1 ng/dL for the control group, at 195±62.2 ng/dL for the octacosanol-administered group, at 299.9±96.6 ng/dL for the chia seed-administered group, at 466.2±44.2 ng/dL for the maca-administered group, and at 617.1±78.5 ng/dL for the group co-administered with chia seeds and maca. As can be seen, the highest level of testosterone was detected in the group co-administered with chia seeds and maca (FIG. 10). The increase of testosterone levels in the test groups, compared to the control group, is thought to result from the fact that chia seeds and maca stimulate the release of testosterone from the testicles. Therefore, the rats administered with the active ingredients were improved in sexual function as their testosterone levels were increased.

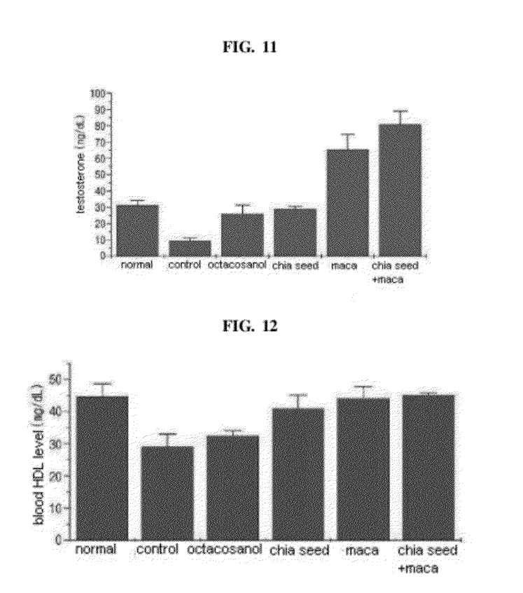

FIG. 11 shows levels of testosterone in blood samples taken from the tail vein in a pattern similar to that in the cardiac blood samples.

To investigate the effect of chia seeds and maca (extract), used as active ingredients in the composition of the present invention, on penile function, the levels of high-density lipoproteins were analyzed. The values were measured at 44.7±4.0 mg/dL for the normal group, at 29.1±3.8 mg/dL for the control group, at 32.5±1.5 mg/dL for the octacosanol-administered group, at 41.0±4.1 mg/dL for the chia seed-administered group, at 44.1±3.6 mg/dL for the maca-administered group, and at 45.2±0.6 mg/dL for the group co-administered with maca and chia seeds. Only the group co-administered with chia seeds and maca was higher in HDL level than was the normal group (FIG. 12). The increase of blood HDL level makes endothelial cells functionally smooth. In addition, the increased HDL level prevents platelet aggregation which in turn inhibits the formation of fibrins, which may promote blood circulation. These effects, together with the enhancement of the hepatic function and the development of the smooth muscle around the corpus cavenosum, are thought to induce a strong erection and increase erection endurance.

### Test Example 9

### Clinical Test

For clinical tests, 20 male smoking subjects suffering from sexual, gastrointestinal or hepatic functions were administered two or three times a day for one month with the composition prepared in Example 8.

Most of the subjects did not experience the sensation of fatigue and enervation which had remained in the subjects administered with chia seeds alone. The subjects co-administered with chia seeds and maca were observed to have more active sensation and to be improved in sexual and gastrointestinal functions, endurance and hangover remedy. More weight loss was detected in the co-administered subjects than in the chia seed-administered subjects. In addition, the co-administration was found to enhance immunity as the subjects experienced rapid wound healing and did little catch a cold. Detailed improved symptoms are summarized in Table 3, below.

TABLE 3
Clinical Test Results

| | No. of experienced |
|---|---|
| Improved Symptom | subject |
| Hangover | 20 |
| Breath care and body odor | 20 |
| Nicotine removal | 20 |
| Gastroenteritis | 19 |
| Sensation of incomplete | 20 |
| urination | |
| Insomnia | 18 |
| Fatigue resistance | 20 |
| Concentration | 19 |
| Inflammation/immunity | 16 |
| Erectile dysfunction | 20 |
| Weight loss | 20 |

As is proven above, co-administration of chia seeds and maca brings about excellent improvement in sexual, gastrointestinal and hepatic functions and endurance. Therefore, the composition comprising chia seeds and maca (extract) in accordance with the present invention may be useful as a pharmaceutical composition as well as a dietary supplement or a feed.

The pharmaceutical composition may vary in dose depending on the patient's sex and age, and the severity of disease. Its daily dose may range from 1.0 g to 50 g when chia seeds and maca are not used in the form of extracts in water or organic solvents, but used as they are. In the case of extracts, the pharmaceutical composition is administered at a daily dose of from 10 mg to 1,000 mg once or many times in a day. However, the present invention is not limited to this regimen.

Also, the pharmaceutical composition may be used in combination with the preexisting agents for sexual and hepatic dysfunction, and endurance, such as Viagra, Levitra, Cialis, heavy drug, Zydena, and may further comprise one selected from the group consisting of green tea, a nucleic acid complex, an alga-derived physiologically active substance, a squeeze of oyster mushroom, a zhibeisan extract, micrococin and a combination thereof. Viagra shows the side effects of increasing NO (nitric oxide) levels in the corpus cavenosum and blood pressure. When used in the combination of the pharmaceutical composition of the present invention, such preexisting agents may be reduced in dose so that their side effects can be alleviated.

The pharmaceutical composition of the present invention may be formulated with pharmaceutically acceptable excipients, auxiliary agents, analgesics, isotonics, and/or preservatives into various dosage forms including injections, liquids, tablets, capsules, powders, and syrups.

The present invention is also described in detail in the following Formulation Examples.

### Formulation Example 1

### Injection 1

In distilled water for injection, 50 mg of the extract from chia seeds and maca, prepared in Example 1, was dissolved to form a total volume of 2 mL, and the solution was adjusted to a pH of 7.6 with 0.1 N NaOH, loaded into a 2 mL ampule, and sterilized to yield an injection.

### Formulation Example 2

### Injection 2

In distilled water for injection, 50 mg of the extract from chia seeds and maca, prepared in Example 2, was dissolved to form a total volume of 2 mL, and the solution was adjusted to a pH of 7.2 with 0.1 N NaOH, loaded into a 2 mL ampule, and sterilized to yield an injection.

### Formulation Example 3

### Tablet 1

Two hundred mg of the extract from chia seeds and maca, prepared in Example 1, was mixed with 100 mg of lactose, 100 mg of starch and a suitable amount of magnesium stearate and pressurized into a tablet using a typical tableting method.

### Formulation Example 4

### Tablet 2

Ten mg of the extract from chia seeds and maca, prepared in Example 1, was mixed with 100 mg of lactose, 50 mg of starch and a suitable amount of magnesium stearate and pressurized into a tablet using a typical tableting method.

### Formulation Example 5

### Powder

One thousand mg of the extract from chia seeds and maca, prepared in Example 5, was mixed with 100 mg of lactose and 2 mg of magnesium stearate, and the mixture was packed in polyethylene chloride-coated sheets and sealed to provide powder.

### Formulation Example 6

### Capsule 1

Five hundred mg of the extract from chia seeds and maca, prepared in Example 1, was mixed with 50 mg of lactose, 50 mg of starch, 2 mg of talc and a proper amount of magnesium stearate and loaded to a gelatin capsule according to a typical method to produce a capsule.

### Formulation Example 7

### Capsule 2

Four hundred mg of the extract from chia seeds and maca, prepared in Example 1, was mixed with 100 mg of lactose, 93 mg of starch, 2 mg of talc and a proper amount of magnesium stearate and loaded to a gelatin capsule according to a typical method to produce a capsule.

### Formulation Example 8

### Capsule 3

Two hundred mg of the extract from chia seeds and maca, prepared in Example 1, was mixed with 20 mg of sulfasalazine, 100 mg of lactose, 100 mg of starch, and a proper amount of magnesium stearate and loaded to a gelatin capsule according to a typical method to produce a capsule.

### Formulation Example 9

### Capsule 4

Five hundred mg of the extract from chia seeds and maca, prepared in Example 1, was mixed with 50 mg of sodium alendronate, 100 mg of lactose, 50 mg of starch, 2 mg of talc and a proper amount of magnesium stearate and loaded to a gelatin capsule according to a typical method to produce a capsule.

### Formulation Example 10

### Capsule 5

Five hundred mg of the extract from chia seeds and maca, prepared in Example 1, was mixed with 20 mg of Aricept, 100 mg of lactose, 50 mg of starch, 2 mg of talc and a proper amount of magnesium stearate and loaded to a gelatin capsule according to a typical method to produce a capsule.

### Formulation Example 11

### Capsule 6

Five hundred mg of the extract from chia seeds and maca, prepared in Example 1, was mixed with 10 mg of raloxifene, 50 mg of lactose, 50 mg of starch, 2 mg of talc and a proper amount of magnesium stearate and loaded to a gelatin capsule according to a typical method to produce a capsule.

### Formulation Example 12

### Liquid

Distilled water was added to a mixture of 500 mg of the extract from chia seeds and maca of Example 1, 1000 mg of pamidronate disodium, 20 g of sugar, 20 g of isomerose and a proper amount of a lemon flavorant to form a total volume of 100 mL which was then loaded to a 100 mL brown vial and sterilized to produce a liquid agent.

Although the embodiments of the present invention have been disclosed for illustrative purposes, it will be appreciated that the present invention is not limited thereto, and those skilled in the art will appreciate that various modifications, additions and substitutions are possible, without departing from the scope and spirit of the invention. Accordingly, any and all modifications, variations or equivalent arrangements should be considered to be within the scope of the invention, and the detailed scope of the invention will be disclosed by the accompanying claims.

1. A composition comprising chia seeds and maca at a weight part ratio of 100:5˜250.

2. The composition according to claim 1, wherein the chia seeds and maca are in a form of an extract, said extract being prepared using an extraction solvent selected from the group consisting of water, ethanol and a mixture thereof.

3. The composition according to claim 1, further comprising a mineral selected from the group consisting of germanium, selenium, dietary zinc and a mixture thereof.

4. The composition according to claim 3, wherein the mineral is contained in an amount from 0.1 to 10 parts by weight based on 100 parts by weight of chia seeds.

5. A pharmaceutical composition for enhancement of sexual, gastrointestinal or hepatic functions, comprising chia seeds and maca at a weight part ratio of 100:5˜250 as an active ingredient.

6. A dietary supplement comprising the composition of claim 1.

7. A feed comprising the composition of claim 1.

8. A preparing method for a food, a feed or a pharmaceutical composition, the method comprising the steps of:
(A) mixing 100 parts by weight of chia seeds with 5˜250 parts by weight of maca in 500˜7000 parts by weight of a solvent selected from the group consisting of water, ethanol and a mixture thereof, based on 100 parts by weight of chia seeds;
(B) heating the mixture of chia seeds and maca in the solvent at a temperature of 30˜60° C. for 2˜6 hours to afford an extract; and
(C) subjecting the extract to filtration and concentration to dryness.

9. A preparing method according to claim 8, further comprising the step (D) of pulverizing the chia seeds and the maca into a powder with a mean particle size of 0.3˜2 μm prior to the step (A).

10. A food additive comprising the composition of claim 1.

11. A food additive comprising the composition of claim 2.

12. A feed additive comprising the composition of claim 1.

13. A feed additive comprising the composition of claim 2.

14. A dietary supplement comprising the composition of claim 2.

15. A feed comprising the composition of claim 2.
